(19) United States
(12) Patent Application Publication
(10) Pub. No.: US 2017/0164954 A1
(43) Pub. Date: Jun. 15, 2017

(54) FLEXIBLE TAP

(71) Applicant: IntraFuse, LLC, Logan, UT (US)

(72) Inventor: Mark D. Hayes, Smithfiled, UT (US)

(73) Assignee: IntraFuse, LLC, Logan, UT (US)

(21) Appl. No.: 15/366,445

(22) Filed: Dec. 1, 2016

Related U.S. Application Data
(60) Provisional application No. 62/266,009, filed on Dec. 11, 2015.

(51) Int. Cl. A61B 17/16

(52) U.S. Cl. CPC A61B 17/1655; A61B 17/1637; A61B 17/1642; A61B 2017/564

(57) ABSTRACT

Examples of the present invention relate to a flexible tap and method for forming a thread in bone. In one example, the tap threads a curved hole in a bone for receiving a flexible threaded component.

### CROSS-REFERENCE TO RELATED APPLICATIONS

This application claims the benefit of U.S. Provisional Application No. 62/266,009, filed Dec. 11, 2015.

### FIELD OF THE INVENTION

Examples of the invention relate to a flexible tap and method for forming a screw thread in a bone.

### BACKGROUND

Orthopedic medicine provides a wide array of implants that can be engaged with a bone such as for example to replace a portion of the bone or immobilize a fracture. It is common to utilize threaded components to engage the bone and to form a thread in a bone to receive the threaded components. Prior art surgical instruments are limited to forming threads along straight paths in bones. However, it would be advantageous to form a thread along a curved path in a bone such as for example to maximize the length of engagement with the bone or to follow a curved portion of the bone such as for example an intramedullary canal. What is needed is a way to form a thread in a bone along a curved path.

### SUMMARY

Examples of the invention provide a flexible tap for forming a thread in a bone.

In one example of the invention, the tap forms a thread in a curved hole for receiving a flexible threaded component.

In another example of the invention, a bone tap includes a first member including a tap head, an elongated flexible shaft, and a driving shaft. The tap head includes a tooth operable to form a thread in bone. The elongated flexible shaft has a first end connected to the tap head and a second end opposite the first end. The driving shaft has a first end connected to the second end of the flexible shaft and includes a thread having a thread pitch. The bone tap includes a second member threadably engaged with the driving shaft and having a bone engagement portion.

In another example of the invention, a method of forming a thread in a bone includes engaging a tap with a bone to fix an anchor member of the tap relative to the bone, the anchor member including a thread having a thread pitch; and rotating a driving shaft relative to the anchor member, the driving shaft being threadably engaged with the anchor member to advance the driving shaft a distance equal to the thread pitch with each full rotation of the driving shaft, the driving shaft driving a tap head to form a thread in the bone, the thread in the bone having a thread pitch corresponding to the thread pitch of the anchor member.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an isometric view of a flexible tap according to one example of the invention;

FIG. 2 is a detail view of the tap of FIG. 1;

FIG. 3 is an end view of a tap head of the tap of FIG. 1;

FIG. 4 is a detail view of the tap of FIG. 1;

FIG. 5 is a side elevation view of the tap of FIG. 1;

FIG. 6-7 are partial section views of the tap of FIG. 1; and

FIGS. 8-9 illustrate the use of the tap of FIG. 1 to form a thread along a curved path in a bone.

### DESCRIPTION OF THE ILLUSTRATIVE EXAMPLES

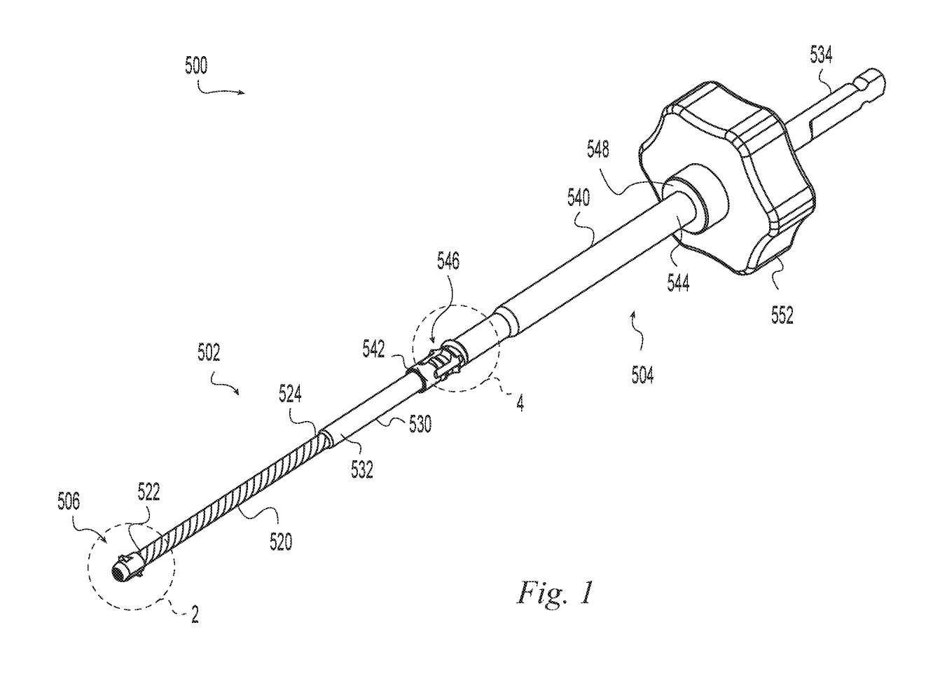

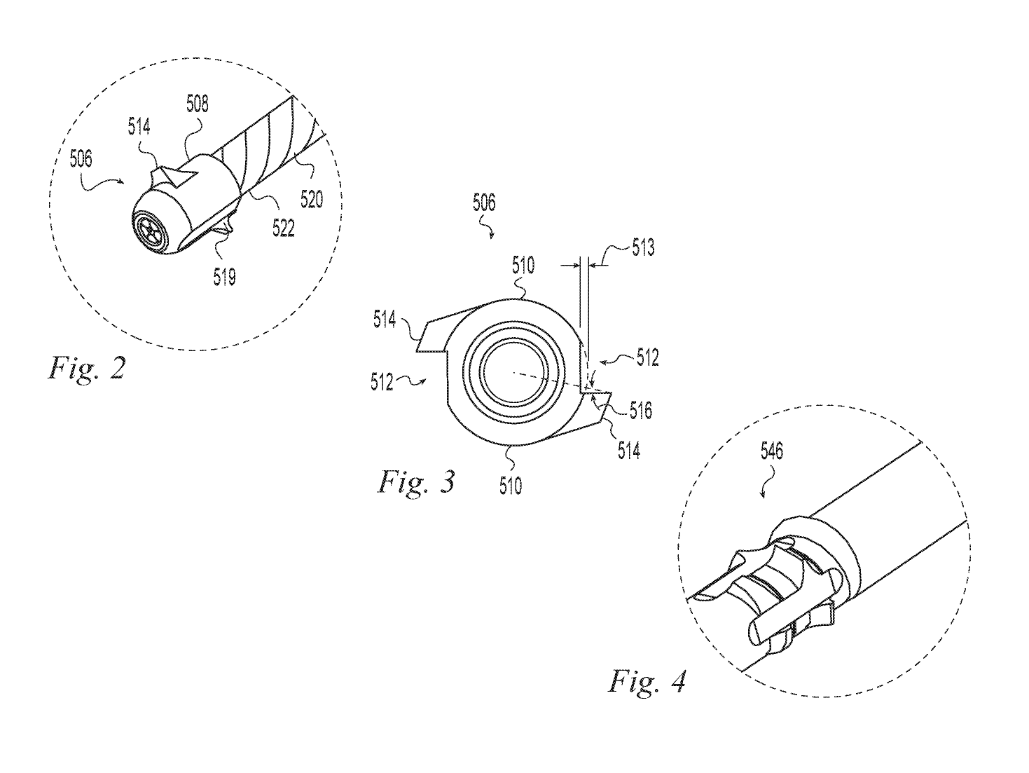

FIGS. 1-7 depict an illustrative example of a flexible tap 500 according to the present invention. The tap 500 is capable of forming a thread along a straight or curved path. For example, the tap 500 is capable of forming a thread along a curved path in a bone to receive a threaded component.

The tap 500 includes a first member 502 and a second member 504 engaged with the first member 502. The first member 502 can be rotationally driven relative to the second member so that the first member advances a predetermined amount with each full rotation of the first member 502. The first member includes a thread forming portion that forms a thread in a bone as it is advanced relative to the second member. At least a portion of the first member 502 is flexible so that the cutting portion can follow a curved path in the bone.

In the illustrative example of FIGS. 1-7, the first member 502 includes a tap head 506 having a generally cylindrical body 508. The body 508 includes a pair of opposing lands 510 and intervening flutes 512 having a flute depth 513. A screw thread segment projects from each land 510 to form a tooth 514 having a tooth face 519. The tooth 514 is adapted to form a thread in bone. The tooth 514 may deform or cut the bone to form the thread. In the illustrative example of FIGS. 1-7, the tooth 514 is adapted to cut a thread in a bone. The face 519 is angled away from a radial reference line toward the center of the tap head to create a positive rake angle 516. The face 519 projects a desired thread profile for that tooth to form into the bone. The tap head 506 may have a single tooth operable to single point cut a spiral thread in the bone as the tap head is rotated. Alternatively, the tap head 506 can have a two or more teeth such as shown in the illustrative example of FIGS. 1-7. However, the tap head 506 is intended to be able to follow a curved path in a bone. As the tap head 506 follows a curved path to form a thread about the path axis, the pitch of the thread so formed will vary from a minimum on the inside of the curve to a maximum on the outside of the curve. With an increasing number of teeth 514, and especially as the number of thread segments along the length of the tap head 506 is increased, the tap head 506 becomes more constrained. Driving a tap head with a large number of teeth along a curved path will result in damage to the formed bone thread due to e.g. the trailing teeth interfering with the bone thread as the leading teeth cause the tap head to tilt to follow the curved path. A single tooth provides the least constraint and the greatest ease in following a curved path. Two teeth, as in the illustrative example of FIGS. 1-7, may help balance the loads on the tap head while still allowing sufficient maneuverability to produce a well formed thread. Also, with two teeth, the leading tooth may project a shorter distance 518 from the land 510 so that a portion of the thread depth is removed by the first tooth and another portion is removed by the second tooth to reduce the torque required to drive the tap. When used to tap a pre-drilled hole, the land, or lands, fit within the hole and guide the tap head 506 along the hole while the teeth 514 cut the thread into the bone.

In the illustrative example of FIGS. 1-7, the first member 502 further includes an elongated flexible shaft 520 having a first end 522 connected to the tap head 506 and a second end 524 opposite the first end. The flexible shaft 520 may include a variety of flexible constructs as is known in the art such as, for example, twisted cables, helical cut tubes, interlocking tongue and groove segments, and other flexible constructs. In the illustrative example of FIGS. 1-7, the flexible shaft 520 includes a twisted cable construction with an inner cable twisted in a first direction and an outer cable twisted in an opposite direction to provide torque transmitting capability in both rotational directions.

In the illustrative example of FIGS. 1-7, the first member further includes a driving shaft 530 having a first end 532 connected to the second end 524 of the flexible shaft and a second end 534 opposite the first end 532. The driving shaft 530 includes a helical thread 536 having a thread pitch 538. In the illustrative example of FIGS. 1-7, the thread 536 is a multi-lead thread having two separate thread flights 537, 539 intertwined along the driving shaft 530 and the thread pitch 538 of each thread flight is measured as shown at reference numeral 538. The thread pitch 538 is the distance the driving shaft 530 will translate along its axis for each complete revolution of the driving shaft 530. Where the tap head 506 includes multiple teeth 514, the teeth are spaced longitudinally a distance corresponding to the driving shaft thread pitch 538. Preferably the driving shaft is rigid. Also preferably, the second end 524 includes an engagement portion releasably engageable with a driver. A driver may be a handle to provide a grip for manually turning by a user or a driver may be a rotary mechanism such as a powered drill.

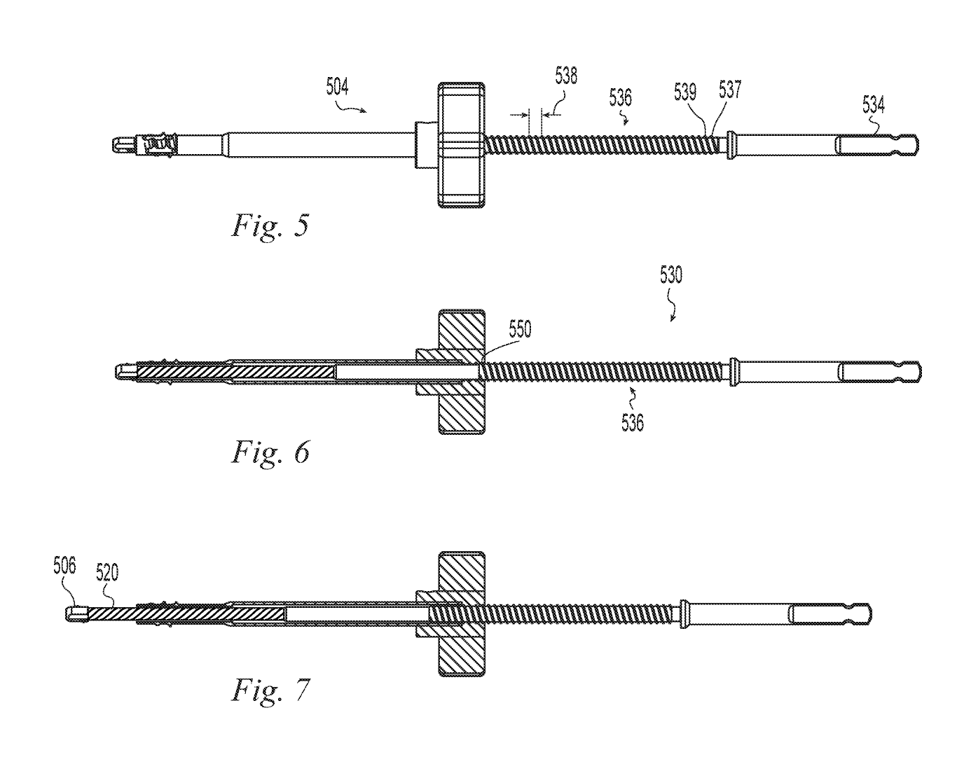

In the illustrative example of FIGS. 1-7, the second member 504 is threadably engaged with the thread 536 of the driving shaft 530 such that rotating the driving shaft 530 relative to the second member 504 translates the driving shaft 530 and consequently the flexible shaft 520 and tap head 506 a distance equal to the thread pitch 538 with each revolution of the drive shaft 530. The tap head 506 will form a thread in a bone with a pitch equal to the driving shaft thread pitch 538. Changing the driving shaft thread pitch 538 will change the formed bone thread pitch to a corresponding value. In the illustrative example of FIGS. 1-7, the second member 504 is an anchor member able to be anchored to a bone and includes a hollow shaft 540 having a first end 542 and a second end 544 opposite the first end. The first end 542 defines a bone engagement portion having an anchor feature that grips the bone to secure the second member 504 against axial translation relative to the bone as the drive shaft 530 is rotated and the bone is threaded. In other words the anchor feature provides a counterforce to allow the threaded engagement between the first and second members to drive the tap head 506 into the bone. The anchor feature may include barbs, threads, pins, screws, expandable members and other suitable features for securing a member to a bone. In the illustrative example of FIGS. 1-7, the anchor feature includes a self-tapping thread 546 formed on the first end 542 of the shaft 540. The second end 544 of the shaft is joined to a hub 548 having a threaded bore 550 (FIG. 6) engaged with the thread 536 of the driving shaft 530. A knob 552 is mounted to the hub 548 to facilitated engaging the self-tapping thread 546 with a bone. FIGS. 6 and 7 are partial sectional views depicting the second member 504 in cross section and the first member 502 in orthographic projection to show the interaction between the two. As seen in FIG. 6, the thread 536 is engaged with the bore 550. In FIG. 7, the driving shaft 530 has been rotated four revolutions to advance the driving shaft 530, flexible shaft 520, and tap head 506 four pitch lengths relative to the second member 504.

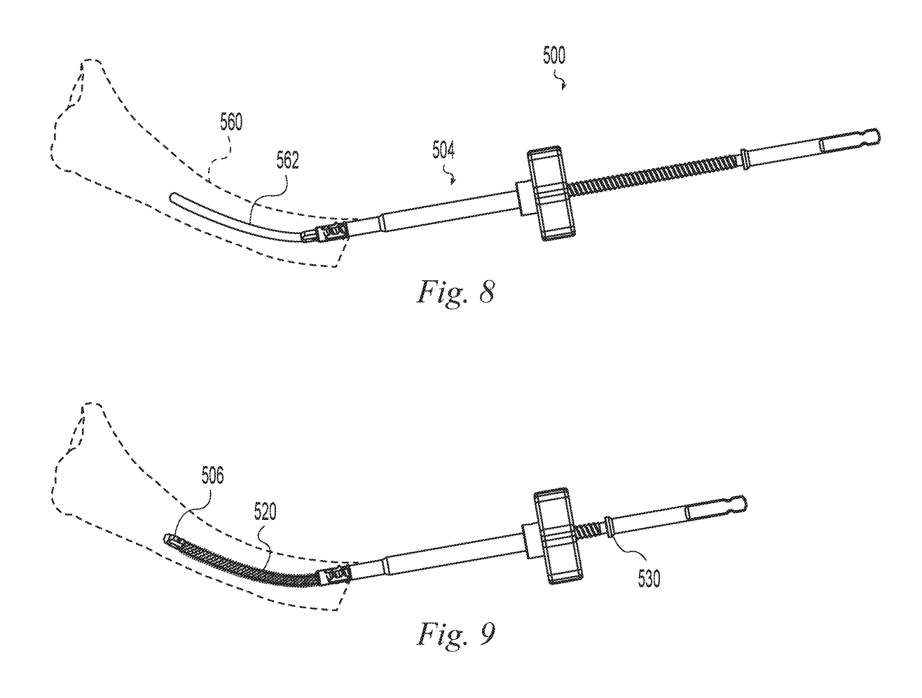

FIGS. 8 and 9 depict an illustrative example of a method of forming a thread in a bone 560 using the tap 500 of FIGS. 1-7. A path for the tap 500 is defined in the bone 560. The path may be defined by a natural bone feature such as an intramedullary canal. The path may be defined by introducing a guide wire in the bone and the tap 500 may be cannulated to follow the guide wire. The path may be defined by forming a hole 562 in the bone 550 as shown in the illustrative example of FIGS. 8 and 9. The path may be straight or curved and the tap 500 may be used for tapping straight or curved holes. However, the tap 500 is particularly useful for forming a thread in curved holes that traditional rigid taps are incapable of tapping. In the illustrative example of FIGS. 8 and 9, the hole is curved such as might be produced by flexibly reaming an intramedullary canal of a curved bone such as a clavicle, rib, or other curved bone.

In FIG. 8, the tap is engaged with the hole 562 by turning the anchor feature of the second member 504 into the hole 562.

In FIG. 9, the driving shaft 530 has been rotated several revolutions to advance the tap head 506 into the bone hole to form a thread in the bone having a pitch equal to the driving shaft thread pitch 538. While the driving shaft 530 is preferably rigid and advances linearly relative to the second member 504, the flexible shaft 520 bends so that the tap head 506 may follow any curvature in the path defined in the bone.

Various examples have been illustrated and described. The various examples may be substituted and combined and other alterations made within the scope of the invention. For example, the depiction of male and female part engagements may be reversed.

What is claimed is:

1. A bone tap comprising:
a first member including a tap head having a tooth operable to form a thread in bone, an elongated flexible shaft having a first end connected to the tap head and a second end opposite the first end, and a drive thread having a thread pitch; and
a second member threadably engaged with the first member, the second member having a bone engagement portion.

2. The bone tap of claim 1 wherein the first member includes a driving shaft having a first end connected to the second end of the flexible shaft, the driving shaft including the thread.

3. The bone tap of claim 2 wherein the flexible shaft is operable to bend to allow the tap head to follow a curved path in the bone.

4. The bone tap of claim 2 wherein the thread of the driving shaft is a multi-lead thread.

5. The bone tap of claim 2 wherein the driving shaft is rigid.

6. The bone tap of claim 2 wherein the driving shaft includes a second end opposite the first end of the driving shaft, the second end releasably engageable with a driver.

7. The bone tap of claim 2 wherein the bone engagement portion includes a bone engagement feature selected from the group consisting of barbs, pins, screws and expandable members.

8. The bone tap of claim 2 wherein the second member comprises a hollow shaft having an internal thread engaged with the driving shaft thread.

9. The bone tap of claim 8 wherein the bone engagement portion comprises a self-tapping thread formed on a leading end of the hollow shaft.

10. The bone tap of claim 9 wherein the hollow shaft comprises a knob operable to rotate the self-tapping thread into bone.

11. The bone tap of claim 8 wherein the internal thread and the driving shaft thread have a common thread pitch, the driving shaft being operable to advance the tap head a distance corresponding to the thread pitch with each rotation of the driving shaft relative to the second member.

12. The bone tap of claim 1 wherein the tap head has only a single tooth operable to form a spiral thread in the bone as the tap head is advanced relative to the second member.

13. The bone tap of claim 1 wherein the tap head has two teeth operable to form a spiral thread in the bone as the tap head is advanced relative to the second member.

14. A method of forming a thread in a bone, the method comprising:
engaging a tap with a bone to fix an anchor member of the tap relative to the bone, the anchor member including a drive thread having a thread pitch; and
rotating a driving shaft relative to the anchor member, the driving shaft being threadably engaged with the drive thread of the anchor member to advance the driving shaft a distance equal to the thread pitch with each full rotation of the driving shaft, the driving shaft driving a tap head to form a thread in the bone, the thread in the bone having a thread pitch corresponding to the drive thread pitch.

15. The method of claim 14 wherein the tap head is connected to the driving shaft by a flexible shaft and further wherein rotating the driving shaft drives the tap head to follow a curved path in the bone.

16. The method of claim 15 further comprising forming a curved hole in the bone and wherein rotating the driving shaft drives the tap head to follow the curved hole.

17. The method of claim 15 further comprising inserting a wire into the bone to define a curved path and wherein rotating the driving shaft drives the tap head to follow the wire along the curved path.

18. The method of claim 15 wherein the anchor member comprises a hollow shaft having an internal drive thread and the driving shaft has an external thread engageable with the internal drive thread.

19. The method of claim 18 wherein the anchor member includes a bone engagement feature selected from the group consisting of barbs, pins, screws and expandable members.

20. The method of claim 18 wherein the anchor member further comprises an external anchor thread at a leading end and wherein engaging the tap with the bone comprises screwing the external anchor thread of the anchor member into the bone.
